(19) United States
(12) Patent Application Publication
(10) Pub. No.: US 2017/0020715 A1
(43) Pub. Date: Jan. 26, 2017

(54) ORAL APPLIANCE

(71) Applicant: DREAMSCAPE MEDICAL LLC, Atlanta, GA (US)

(72) Inventors: Elijah Charles Walker, Silver Spring, MD (US); Anthony P. Kimani Mwangi, Fairburn, GA (US)

(21) Appl. No.: 15/072,810

(22) Filed: Mar. 17, 2016

Related U.S. Application Data
(62) Division of application No. 14/203,791, filed on Mar. 11, 2014, now abandoned.
Application No. 14/203,791 is a continuation-in-part of application No. 13/412,209, filed on Mar. 5, 2012, now abandoned.
Application No. 13/412,209 is a continuation-in-part of application No. 12/273,534, filed on Nov. 18, 2008, now Pat. No. 8,127,769.
(60) Provisional application No. 61/791,702, filed on Mar. 15, 2013.
(60) Provisional application No. 60/988,794, filed on Nov. 18, 2007.

(51) Int. Cl. A61F 5/56; A61M 16/20

(52) U.S. Cl. CPC A61F 5/566; A61M 16/20; A61M 2230/00

(57) ABSTRACT

An integrated oral appliance for treating breathing obstruction, snoring and restriction of the upper airway during sleep with a tongue restraint. Oral appliances of the invention may include an oral cavity engagement device including one or more trays, a tongue restraint with a tongue contact portion, a spring force element operatively coupled between the at least one tray and the tongue restraint, wherein the spring force element is adjustable anteriorly and posteriorly relative to the tray(s), and an air conduit inside the tongue restraint with a front vent of the air conduit extending beyond an anterior end of the tray(s) and a rear opening of the air conduit at a posterior end of the of the oral cavity engagement device configured to extend to a posterior surface of a tongue.

### CROSS-REFERENCE TO RELATED APPLICATIONS

The present application is a division of U.S. patent application Ser. No. 14/203,791, filed Mar. 11, 2014, which claims benefit of priority to U.S. provisional application 61/791,702, filed Mar. 15, 2013, and is a continuation-in-part of U.S. patent application Ser. No. 13/412,209, filed Mar. 5, 2012, now abandoned, which is a continuation-in-part of U.S. patent application Ser. No. 12/273,534, now U.S. Pat. No. 8,127,769, filed Nov. 18, 2008, which claims the benefit of priority of U.S. provisional patent application No. 60/988,794, filed Nov. 18, 2007, all of which are incorporated by reference herein.

### FIELD OF THE INVENTION

The present invention application pertains to oral (i.e. intraoral) appliances for treating, monitoring and reporting sleep-related breathing disorders, as well as non-sleep related disorders. More particularly, this invention application pertains to oral appliances that use multiple methods to enhance breathing during sleep by preventing and alleviating upper airway obstruction and restriction resulting from sleep-related breathing disorders such as snoring, obstructive sleep apnea, obstructive sleep hypopnea, or upper airway resistance. This invention also pertains to oral appliances that monitor and report information and oral appliances that can provide substances such as medication and or nutritional substances.

### BACKGROUND OF THE INVENTION

Sleep is a fundamental need and appears to be required for human survival. However, for many people diagnosed with sleep apnea, going to sleep can be a dreaded experience due to the lack of restful sleep. Sleep apnea (apnea meaning a cessation of airflow) is a relatively common and potentially life-threatening sleep disorder that impacts millions of people in the United States and around the world.

Obstructive sleep apnea (OSA), obstructive sleep hypopnea, and upper airway resistance are characterized by upper airway abnormalities that result in airway collapse and complete or partial obstruction of airflow into the lungs. Upper airway (i.e. upper respiratory tract, or airway) abnormalities include: a smaller (than normal) airway cross-sectional area that subjects the pharynx to collapse; an enlarged tongue that can obstruct the airway by moving posteriorly (backward) into airway space during sleep; a retruding jaw that can increase tissue pressure surrounding the airway and subject it to collapse; an enlarged soft palate that can impinge on airway space when breathing; or compromised pharyngeal dilator muscles that fail to keep the airway open when inhaling, causing momentary obstruction of airflow. Fortunately, the brain usually detects this inability to breathe and briefly awakens the individual to reopen the airway. Unfortunately, these continuous disruptions in breathing have also been associated with increased blood pressure, stroke, and diabetes as well as other chronic disorders including death. Due to the variety of airway abnormalities that cause obstruction, and individual needs and preferences, no single solution has been found to be acceptable to all who suffer from OSA.

Forced Ventilation Approaches

Various apparatus-based approaches (e.g. non-surgical and non-pharmacological) have been developed to treat snoring and/or sleep apnea which in general can be divided into two categories: 1. apparatus that require a power source and 2. apparatus that do not require a power source. Airway Pressure apparatus that require power sources (usually involving forced ventilation) include medical devices, such as Continuous Positive Airway Pressure (CPAP) devices, and negative pressure apparatus. Although CPAP devices have success rates of approximately 82.7% (Hoekema A, et al, “Obstructive Sleep Apnea Therapy”, J Dent Res. 2008 September 87(9):882-7), they suffer from low user compliance estimated to be approximately “25-50% with patients typically abandoning therapy during the first 2 to 4 weeks of treatment”; (Zozula, R. et al, Compliance with continuous positive airway pressure therapy . . . ”, Current Opinion in Pulmonary Medicine. 7 (6): 391-398, November 2001). Those who dislike CPAP give many reasons including: mask discomfort, difficulty adapting to the pressure, dislike being tethered to a machine, nasal irritation, sore throat, and allergies.

Current Oral Appliance Approaches

Current oral appliance approaches offer additional solutions for snoring or sleep apnea. Oral appliances can generally be separated into two types: Mandibular Repositioning Appliances, (e.g. U.S. Pat. No. 6,729,335, Halstrom, May 4, 2004) and Tongue Retainer appliances. Mandibular Repositioning Appliances (MRAs, sometimes known as mandibular advancement appliances) purport to reposition the mandible anteriorly to further open the airway to prevent its obstruction.

Traditional Tongue Retainer (TR) appliances are not used very often, and in general, they either use a medium such as a suction to hold/pull the tongue or they use a direct contact device to hold/restrain the tongue during sleep. Suction-type TRs may be recommended when users lack adequate teeth or when the lower jaw can't be advanced. One vacuum-type TR, (U.S. Pat. No. 4,676,240, Gardy, Jun. 30, 1987) purports to provide a way to hold the tongue forward in a chamber that generates a vacuum when the tongue begins to fall and also purports to allow oral breathing.

A direct contact type of TR (e.g. U.S. Pat. Application Pub. 2008/0041396 A1, Lucker, Feb. 21, 2008) purports to restrain the tongue using a rigid flat tab at the back of the tongue and uses another rigid tab-like component in the area of the soft palate. Another TR appliance (e.g. U.S. Pat. No. 6,766,802, Keropian, Jul. 27, 2004) purports to hold the tongue down using a rigid bar-like device.

Shortcomings of Prior Art

Prior art offers a variety of purported solutions along with significant drawbacks. Oral appliances (MRA, TR, etc.) in general have treatment success rates of approximately 54%, and compliance rates of 56-68%. (Hoffstein V, “Review of oral appliances for treatment of sleep-disordered breathing”, Sleep Breath. 2007 March; 11(1):1-22). MRA users have experienced mixed success, with some patients experiencing potentially harmful dental changes and/or temporomandibular joint pain. A study reported that with long term use (88.4+/−26.7 months) there were significant dental changes including “mandibular arch width increased more than maxillary arch width” (Chen H, et al, “Three-dimensional . . . Part 2. Side effects of oral appliances . . . ”, Am J Orthod Dentofacial Orthop. 2008 September; 134(3):408-17).

Pre-fabricated thermoplastic (boil and bite) MRAs have been determined to be ineffective and “cannot be recommended as a therapeutic option nor can it be used as a screening tool to find good candidates for mandibular advancement therapy” (Vanderveken, O M, et al, “Comparison of a custom-made and a thermoplastic oral appliance for the treatment of mild sleep apnea.”, Am J Respir Crit Care Med 2008; 178:197-202).

Vacuum-type TR devices that purport to hold the tongue suffer from low compliance rates, reported to be 25% in one study (e.g. 75% non-compliance) and low efficacy i.e. low treatment success, (Schonhofer, B et al, “Value of various intra- and extraoral therapeutic procedures for obstructive sleep apnea and snoring”, Med Klin (Munich, 1997 Mar 15; 92 (3): 167-74 9173209).

Part of the difficulty of restraining or holding the tongue is due to the typically wet, slippery nature of the tongue. Saliva is continuously produced in the oral cavity at a rate of approximately 1.5 liters per day, which increases when foreign objects are added. Additionally, glycoproteins in saliva (which provide its lubricative characteristic), increase the difficulty of holding the tongue.

Thus, there remains a need for a treatment apparatus that does not require forced ventilation, or being tethered, and is effective in restraining intraoral tissue including the tongue and or mandible without adverse effects such as discomfort, pain, tissue dysfunction, or injury.

It would be advantageous to provide an effective oral appliance to maintain upper airway patency during sleep, thus preventing obstructions and snoring without adverse effects.

It would be advantageous to provide an oral appliance to maintain airway patency comprising mandible repositioning and tongue restraint to improve treatment effectiveness and comfort.

It would be advantageous to provide an oral appliance comprising a comfortable, easily adjustable mandible repositioning method.

It would be advantageous to provide an oral appliance comprising bristles and or other materials or means to interact with the tongue without excessive force.

It would be advantageous to provide an oral appliance comprising an air conduit to provide breathable air via the oral cavity while keeping the mouth closed thus bypassing upper airway obstructions and nasal restrictions.

It would be advantageous to provide an oral appliance comprising a means to provide substances such as medications and or nutritional substances.

It would be advantageous to provide an oral appliance that can be located and maintained in the oral cavity using a variety of means.

It would be advantageous to provide an oral appliance that can detect, monitor, and report physiological variables inside the oral cavity.

It would be advantageous to provide an oral appliance that can electromechanically interact with oral cavity tissue.

The aforementioned and other advantages, features and characteristics of the present invention, as well as the methods of operation and functions of the related elements of structure and the combination of parts and economies of manufacture, will be more apparent upon consideration of the following detailed description and claims, with reference to the accompanying drawings; all of which form a part of this specification, wherein like reference numerals designate corresponding elements in the various figures. It should also be understood that the drawings are for the purpose of illustration and description and are not intended to specify the limits of the invention. Nor is the size, scale or orientation of elements shown in the drawings intended to reflect actual size, scale or proportion. Additionally, the method of the present invention includes any description herein of how the apparatus functions or is used, irrespective of whether such description is specifically identified as method disclosure.

### SUMMARY OF THE INVENTION

In accordance with the present invention, a unique oral appliance integrates several novel features to secure the device in the oral cavity, treat, detect, and monitor sleep-related breathing disorders associated with upper airway abnormalities and snoring or other disorders or conditions that can be treated via the oral and or nasal cavity. The appliance is placed in the oral cavity and supported by engaging oral cavity tissue such as upper and or lower dentition, gingiva, or mucosa such as labial and or buccal mucosa and or supported by means external to the oral cavity. The appliance can use a variety of means to engage oral tissue, and/or teeth including without limitation holding, contacting, frictional force, suction, gripping, captivating, and like engagement means. In some embodiments, standard-sized or custom fit trays may engage oral tissue and/or teeth. In other embodiments, standard-sized or custom fit engagement compounds may be utilized to support the positioning of an oral appliance of the present invention.

An oral appliance in embodiments of the invention can be implemented in a variety of ways to treat a variety of conditions and can comprise a means to locate and secure the device in the oral cavity, a means to advance the mandible, a means to interact with the tongue, a means to allow airflow via the oral cavity, a means to humidify and/or heat the oral cavity, and/or a means to provide medication or nutritional substances. A means can also be provided to connect other devices (such as CPAP devices), or power or methods to secure the oral appliance to the user. Adjustable components facilitate movement of the lower tray to advance the mandible to prevent upper airway closure. Other attached components interact with the tongue to block tongue movement or to apply a compressive spring-loaded, or magnetic, or fixed force to the surface of the tongue. Novel interface (such as bristles, non-slip, or anti-slip materials, or active and non-active components) engage the tongue to prevent it from obstructing the airway. A novel air conduit allows breathable air or other fluids to flow (via the oral cavity) to the upper airway, bypassing nasal restrictions and/or airway obstructions and modulates upper airway air pressure. Means is also provided to humidify and or heat the air cavity. Means are also provided to treat other (sleep related or non-sleep related) disorders or conditions using medications, nutritionals, and like substances. Means are also provided to detect, monitor, store and report physiological data involving the oral cavity and or upper airway.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a left sectional view of an anatomical cross-section of a normal human upper airway including nasal cavity, oral cavity, and pharynx with arrows illustrating normal nasal airflow;

FIG. 2 is a left sectional view of an anatomical cross-section illustrating occlusion of the pharynx that can occur during obstructive sleep apnea;

FIG. 3 is a rear perspective view of an integrated oral appliance in an embodiment of the present invention;

FIG. 4 is a rear exploded perspective view of an integrated oral appliance in an embodiment of the present invention;

FIG. 5 is a rear right partial view of an oral appliance illustrating a coupling for providing advancement of a lower tray relative to an upper tray in one embodiment of the present invention;

FIG. 6 is a rear right partial view of an oral appliance illustrating a coupling for providing advancement of a lower tray relative to an upper tray in one embodiment of the present invention;

FIG. 7 is a rear perspective and partial view of a connector between trays of an oral appliance in one embodiment of the present invention;

FIG. 8 is a rear perspective view of an oral appliance in one embodiment of the present invention;

FIG. 9A is a rear perspective view of upper and lower trays of an oral application in one embodiment of the present invention;

FIG. 9B is a top view of an upper tray of an oral appliance in one embodiment of the present invention;

FIG. 10 is a left sectional view of an anatomical cross-section of a human upper airway and oral appliance engaging upper and lower dentition and coupled to a tongue in one embodiment of the present invention;

FIG. 11A is a rear perspective view of an upper tray and lower tray of an integrated oral appliance with a tongue restraint in one embodiment of the present invention;

FIG. 11B is a perspective view of a tongue restraint with a perspective view of air conduit in one embodiment of the present invention;

FIG. 11C is a perspective view of a tongue restraint of an oral appliance in one embodiment of the present invention;

FIG. 12 is a rear perspective view of an upper tray and lower tray of an integrated oral appliance having a connector to a support strap in one embodiment of the present invention;

FIG. 13 is a front perspective view of an assembly comprising an air conduit, humidification means, and adjustable tongue-restraint in one embodiment of the present invention;

FIG. 14 is a front perspective view of a retainer with air conduit in one embodiment of the present invention;

FIG. 15 is a perspective view of a tray to engage dentition that includes an air conduit in one embodiment of the present invention;

FIG. 16 is a left perspective and partial view of a tray of an oral appliance having suction cups, shown in a cross sectional detail view, in one embodiment of the present invention;

FIG. 17 is a perspective view of a spring air conduit and a fluid absorbent material of an oral appliance in one embodiment of the present invention;

FIG. 18 is an oral appliance with electronic components in one embodiment of the present invention;

FIGS. 19A and 19B are perspective views of an electronic means to detect breathing rate within an oral appliance air conduit by sensing a change in light transmission during inhalation (FIG. 19A) and exhaling (FIG. 19B) in one embodiment of the present invention;

FIG. 20 is a schematic view of an optical source and detector mounted within a dental tray 1 that engages a lip as part of a system to determine heart rate and or oxygen saturation in one embodiment of the present invention;

FIG. 21 is a left cross-sectional view of a human upper airway illustrating an oral appliance positioned in the oral cavity, engaging the upper and lower dentition, and interacting with the tongue using electronic means to prevent it from obstructing the airway in one embodiment of the present invention;

FIG. 22 is a relational block diagram illustrating electronic monitoring of sensors of an oral appliance and data collection in one embodiment of the present invention;

FIG. 23 is a relational block diagram of a switch, clock and display for usage monitoring of an oral appliance in one embodiment of the present invention;

FIG. 24 is a relational block diagram of electronic monitoring and data collection of an oral appliance having an electronic actuator control including tongue control, motor, pump, valve, and the like, in one embodiment of the present invention;

### DESCRIPTION OF THE INVENTION

Accordingly, an embodiment of the present invention comprises several unique features designed to overcome upper airway abnormalities experienced by those who suffer from sleep-related breathing disorders such as obstructive sleep apnea and snoring. Specifically, the integrated oral appliance 13 treats disorders such as obstructive sleep apnea and snoring by preventing or mitigating airway closures by advancing the mandible to further open the airway, providing an air conduit 8, engaging the tongue, providing medication to treat sleep apnea or other disorders, and monitoring and reporting device usage as well as other physiological variables. The present invention, as well as a preferred mode of use, objects and advantages, can be understood by referring to the following detailed description when read in conjunction with the accompanying drawings, wherein:

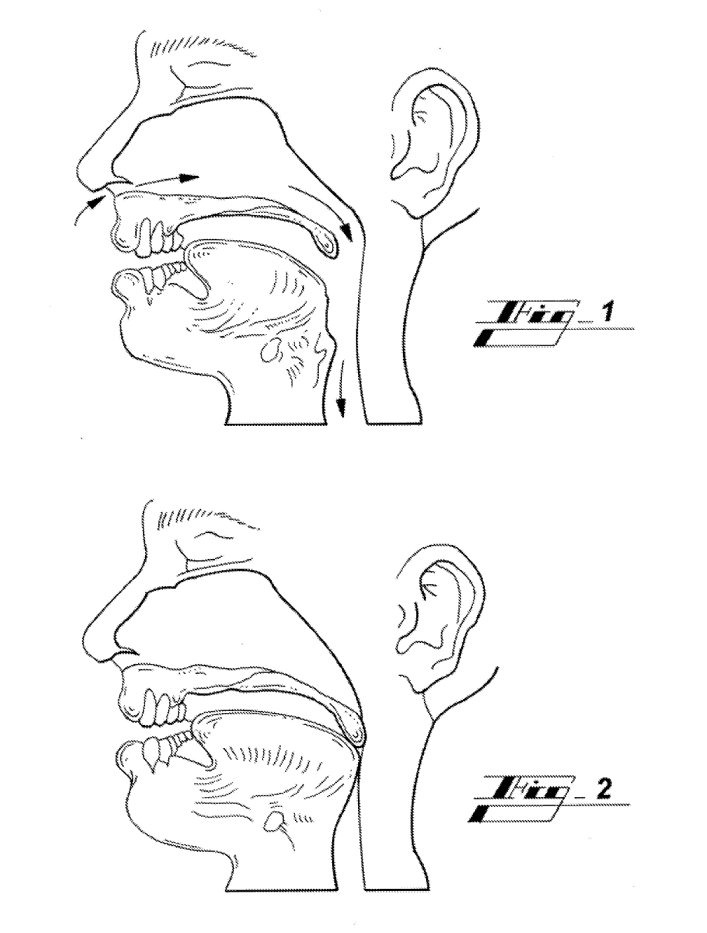

FIG. 1 is an anatomical cross-section of a normal human upper airway including nasal cavity, oral cavity, and pharynx with arrows illustrating nasal airflow;

FIG. 2 is a view similar to FIG. 1, illustrating occlusion of the pharynx that can occur during obstructive sleep apnea;

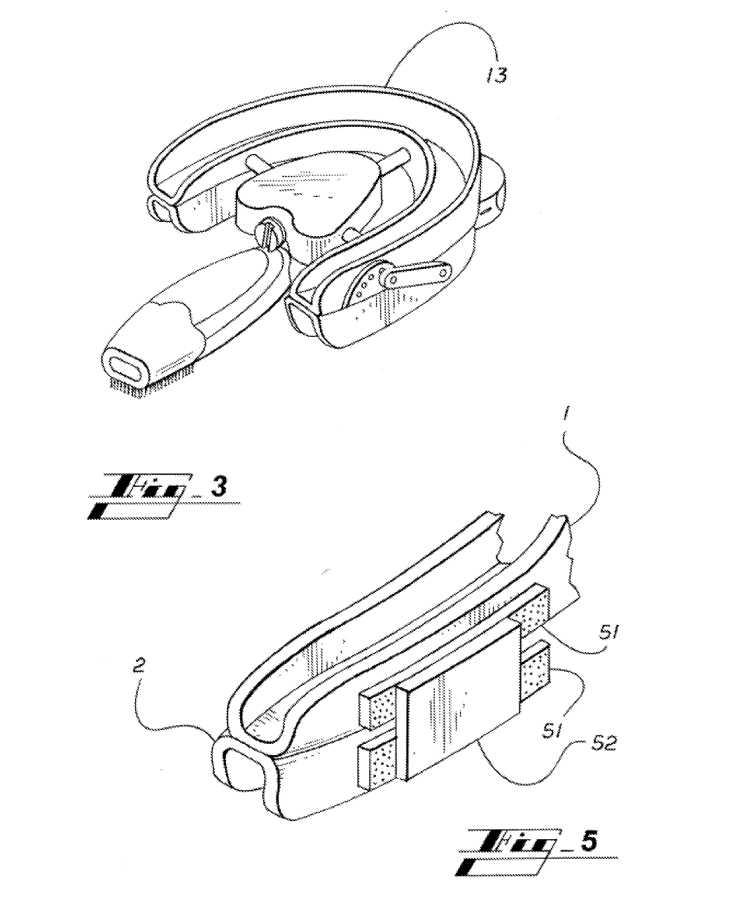

FIG. 3 is a rear perspective view of an integrated oral appliance 13;

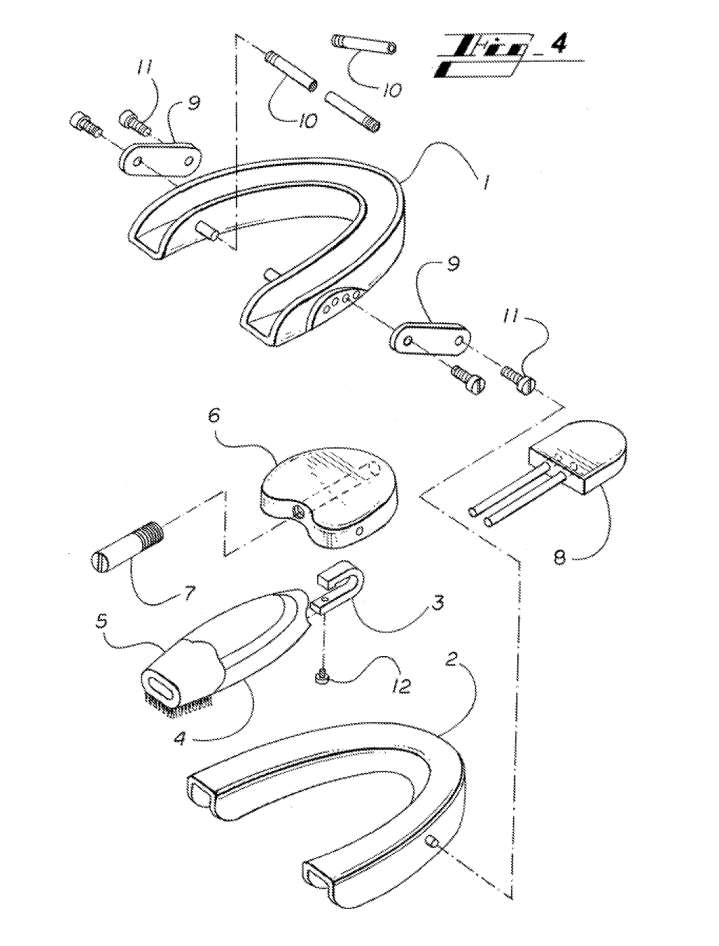

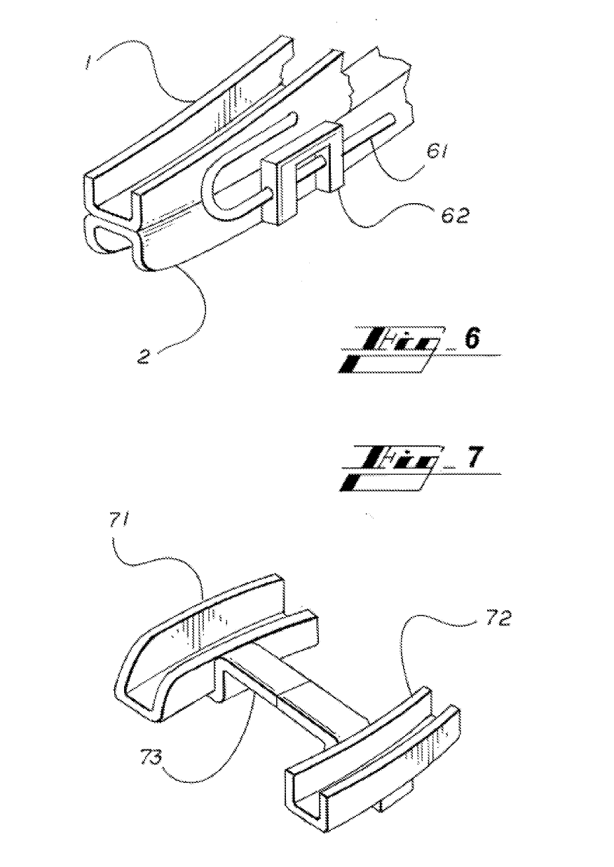

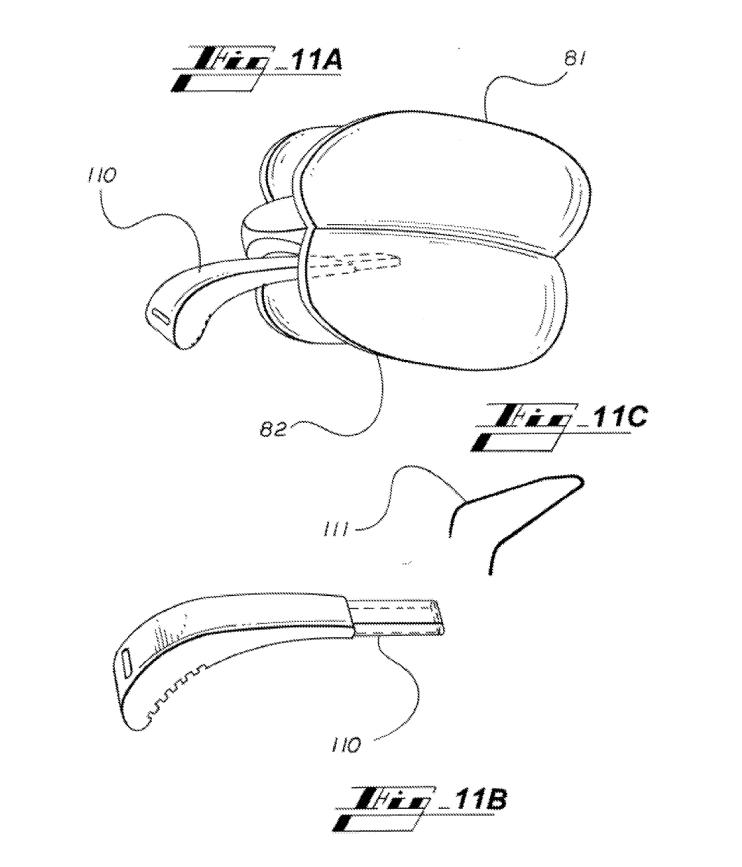

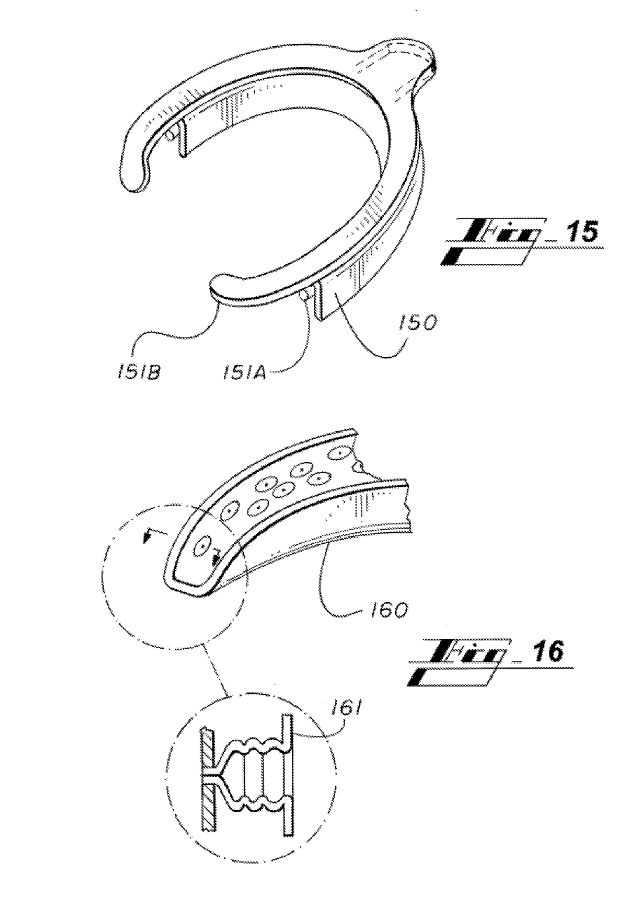

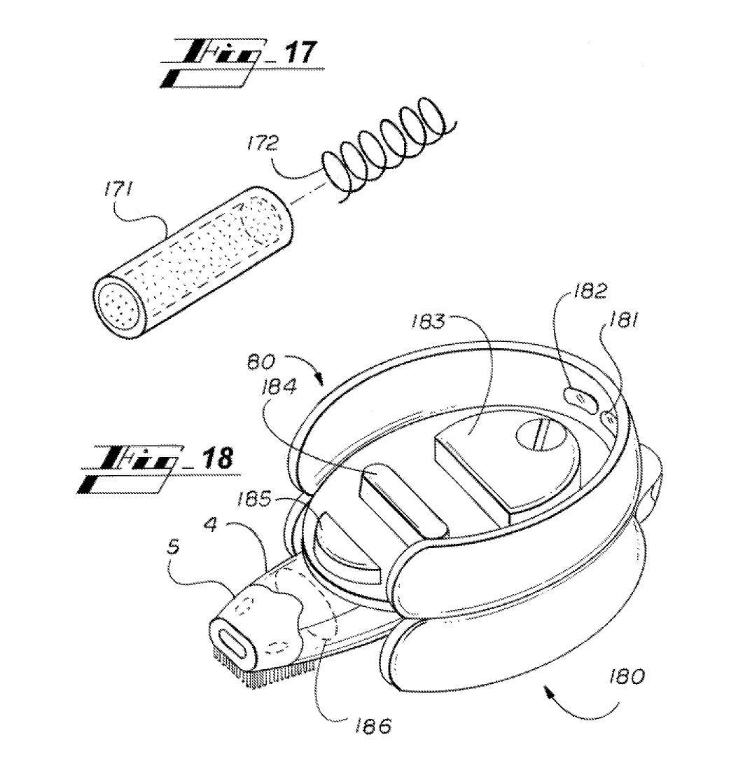

FIG. 4 is a rear exploded perspective view of an integrated oral appliance 13 illustrating the following elements:
i. Element 1 an upper tray to engage dentition and oral tissue comprising a means to connect support elements 10 and a means to provide anterior/posterior adjustment of elements 9 by moving elements 9 to different holes positioned on the sides of element 1;
ii. Element 2 a lower tray to engage dentition and oral tissue comprising a means to connect elements 9 to the lower tray;
iii. Both elements (trays) 1 and 2 can be made from a variety of biocompatible materials (e.g. acrylic, thermoplastics such as ethylene vinyl acetate, thermoplastic elastomer such as Vistamaxx™, ExxonMobil Chemical; or Variflex, Great Lakes Orthodontics, polymeric materials, or medical-grade silicone rubber, or any appropriate material that will functionally perform and will not rust or degrade when exposed to the environment inside the oral cavity. In other embodiments, trays 1 and 2 may facilitate inclusion of additional components such as a built-in air vent or electronic components etc. In other embodiments, elements 1 or 2 may be supported by means exterior to the oral cavity including straps connected via means around the head or neck or ears etc., or clamp to a body surface such as the nose. In other embodiments elements 1 and 2 may be fastened together or constructed as a single unit. In other embodiments, additional means can be used to adjust element 1 relative to element 2 in an anterior/posterior direction such as illustrated in FIG. 5, where hook and loop fastener elements 51 and 52 attached to the outer surface of elements 1 and 2. Alternatively, as illustrated in FIG. 6, a curved rod element 61 may be fixed to element 1 and element 62 adjustably connects element 2 to element 1 to allow element 2 to be adjusted anteriorly relative to element 2. In other embodiments, means can be provided to adjust the vertical spacing between elements 1 and 2. In other embodiments as illustrated in FIG. 7, element 1 and connecting rods 10 can be replaced using dental tray sections, elements 71 and 72 connected with element 73. Elements 71 and 72 can be made using Vistamaxx™, ExxonMobil Chemical. Elements 73 can be made using an appropriate biocompatible rigid material such as stainless steel or polymeric materials etc.
iv. Element 3 provides spring-force to engage the tongue and connects adjustably to element 6 on one end using a tabbed surface that fits into slots on the bottom surface of element 6, and fastened (or fixed) to element 4 on the lower end. In other embodiments, other means can be provided to adjust element 3 in an anterior/posterior direction thus adjusting its position on the tongue. For example, threaded fasteners can also be used. In other embodiments, element 3 can be positionally non-adjustable. In other embodiments, the spring-force range exerted by element 3 can be fixed or adjustable. Element 3 can be fabricated from a material that is biocompatible, lightweight, strong enough to perform said tongue engaging function, and able to endure multiple flexures over the lifetime of integrated oral appliance 13. Element 3 can be made using an appropriate stainless spring steel or a wide variety of other appropriate spring materials including polymeric materials or elastomeric materials, or any appropriate material that will functionally perform and will not rust or degrade when exposed to the environment inside the oral cavity. In other embodiments a variety of spring types can be used such as torsion springs, leaf springs, coiled springs, compression or extension springs, constant force springs, wire-form springs, cantilevered springs, or custom stampings, and the like. In other embodiments a variety of means can be used to provide a spring-force including pneumatics, elastomeric means such as rubber or foam blocks such as in FIG. 11 element 110 or element 111, magnetic means including opposing magnetic fields. In other embodiments a non-spring force, for example a fixed weight or rigid element can be positioned on the tongue. In other embodiments a rigid element can be pressed against the tongue to block its movement as shown in FIGS. 11A-C, element 111 or to compress the tongue using a rigid material. Note that element 111 (FIG. 11C) can be rigid and block tongue movement at the base of the tongue (or other location on the tongue) or provide a spring-force to restrain tongue movement. Note that element 110 (FIGS. 11A and 11B) can be a rigid material or a spring-force material;
v. Element 4 is an enclosure and comprises part of air conduit 8 and is connected to element 3 and supports element 5. Element 4 can be made of an appropriate biocompatible material including polymeric, foam, rubber, stainless steel, etc. In other embodiments element 4 can be open and be an extension of element 3 and provide support for elements 8 and 5. Element 4 can also provide a platform to support electronics, actuators, or medication, etc;
vi. Element 5 serves to engage the tongue and mounts on element 4 (or can be fabricated as a part of element 4) and serves to engage the tongue to mitigate tongue movement. As illustrated, element 5 is made as a band with an array of fiber type projections on the bottom surface that engages the tongue and maintains contact with the tongue under a compressive force that is sufficient to keep the fibers engaged with the tongue to properly restrain it, but not excessive to cause pain or injury. Element 5 may comprise a variety of materials including but not limited to materials such as medical grade or food grade silicone rubber, foam, gel, Vistamaxx™, polymers, polymeric, elastomeric, composite materials, metals, material blends, and the like; or slip-resistant materials, or any appropriate material that will functionally perform and will not rust or degrade when exposed to the environment inside the oral cavity. Element 5 can also be made in a variety of configurations including an array of pores or reliefs, a mesh or irregular or corrugated surface, cavities, a porous matrix, perforations, gratings, and the like;
vii. Element 6 provides support for spring-force element 3 and adjustment of element 3, support for connecting elements 10, support for element 8, and a support means for element 7 to secure element 3 within element 6. Element 6 is fabricated to fit a range of element 1 arch widths using the appropriate size connecting elements 10. Element 6 can also provide a means to mount additional components including electronics or medication supplies;
viii. Element 7 can be fastened into element 6 to secure element 3 within element 6. Element 7 also supports one end of the forward-most element 10. Element 7 can be fabricated using a variety of appropriate materials including stainless steel, or polymeric materials etc;
ix. Element 8 serves as an air conduit that is mounted between elements 1 and 2, and fits between the users lips and connects to element 6 and 4 and 5 to vent air from outside the users lips to inside the oral cavity or inside the upper airway. Airflow within the air conduit 8 occurs naturally as the user inhales and exhales. When the user inhales, a negative pressure is developed within the pharynx (near the end of element 5), causing air to flow from higher pressure (atmospheric pressure) outside the user's oral cavity directly to the user's pharynx. Airflow is reversed when the user exhales. Element 8 can be fabricated using a variety of appropriate materials including elastomers such as silicone rubber, stainless steel, polymeric materials, foam etc. In other embodiments element 8 can be made of fixed length or adjustable length using telescoping sections, or fixed diameter or adjustable diameter, or can be made using a variety of shapes including oval, round, rectangular, square etc, or in other embodiments the air conduit can be perforated to allow moisture to be added to the air. In other embodiments a flow control device (e.g. mechanical or electromechanical valve) can be included to adjust the flow rate of air or gas etc. In other embodiments other features can be included such as a venturi, or one-way valves, or a provision to allow other substances to be conveyed within the air vent such as ports to allow medication etc. In other embodiments additional components can be connected to element 8 to allow forced air devices to be connected. In other embodiments additional connectors can be added to element 8 to allow connection to outside support means such as straps, clamps, etc In other embodiments element 8 may be incorporated as a part of other components such as element 1 or 2, or as a separate device as shown in FIG. 15 where element 150 includes an air vent and a means to engage the tongue. In other embodiments a coiled spring can serve as an air vent when covered by a tube such as illustrated in FIG. 17 where coiled spring 172 is covered by a tube (foam or elastomer). In other embodiments a means to humidify the air can be included such as shown in FIG. 17 where a foam tube element 171 contains a liquid that serves to humidify air flowing through the coiled spring, or the coiled spring can be replaced by a perforated tube. In other embodiments the air conduit can provide multiple functions and serve as a means to engage the tongue as well as a means to convey air. In other embodiments the air conduit 8 can convey other gases or forced air from CPAP devices or other substances such as medication. In other embodiments the air conduit can assist in providing Positive End-Expiratory Pressure (PEEP) through appropriate configuration of valves. In another embodiment a heating element such as resistive element can be provided to heat inhaled air. In another embodiment the air conduit 8 can be eliminated if not required;
x. Elements 9 are rigid links to connect elements 1 and 2 and can be repositioned using elements 11 to move elements 9 to a different hole on the sides of element 1. Elements 9 serve to reposition element 2 thus moves the users mandible. Element 9 can be made using a variety of appropriate materials including stainless steel or polymeric materials;
xi. Element 10 are support rods to connect element 6 to element 1 (or element 2). The rods are sized to connect a wide variation in element 1 arch widths. The rigid rods can be screwed into the interior face of element 1. The rods can be fabricated using a variety of biocompatible materials including stainless steel, or polymers etc Although (3) element 10 rods are illustrated to connect to element 6, other embodiments can comprise connections to element 1 using one or two connecting elements to connect to elements 1 or 2 and support additional components. In other embodiments elements 10 can be made using a single piece of stainless spring steel that forms a bridge that is connected to (2) inner surfaces of element 1 and is appropriately shaped to form a curved cantilever spring to engage the tongue, and support element 8;
xii. Element 11 are fasteners to connect elements 9 to elements 1 and 2 and can be made using a variety of appropriate materials such as stainless steel, or nylon, or other polymeric materials;
xiii. Element 12 is a fastener used to connect one end of element 3 to an end of element 4. Element 4 can be made using a variety of appropriate materials such as stainless steel, or nylon, or other polymeric materials. In other embodiments element 12 can be another type of fastener or can be eliminated if element 3 is built into element 4.

In operation, an appropriate medical professional (e.g. physician, dentist, etc.) can initially fit and adjust the integrated oral appliance 13 to the user oral cavity. Initial one-time adjustments can include mandible repositioning and positioning of tongue-restraint assembly, components 3, 4, 5, to engage the user's tongue. The mandible is moved (repositioned) by moving the part of element 9 that is attached to the side of element 1 to a different hole location on element 1 of FIG. 4. The tongue restraint (or tongue stabilizer (assembled elements 3, 4, 5) is positioned on the tongue by removing element 7 and then moving element 3 within element 6 to the proper position within element 6 of FIG. 4 so that the elements 3, 4, 5 are in a new location on the user's tongue.

In daily practice, prior to falling asleep, the user positions the integrated oral appliance 13 inside their oral cavity so that upper-tray 1 and lower-tray 2 engages dentition and the assembly 3, 4, 5 engages the tongue. During sleep, the oral appliance mitigates obstructive sleep apnea and snoring by advancing the mandible (if necessary) to further open the airway, engaging the tongue to prevent it from obstructing the upper airway, allowing an alternative air path (element 8) via the oral cavity which also serves to reduce pharyngeal collapse pressure near the tongue and soft palate, and may treat other conditions via medication. After waking up, the user removes the oral appliance from their oral cavity and cleans it using an appropriate method. If the oral appliance comprises electronics (FIG. 18) the user may also transfer data.

Thus, it can be seen from the above illustrative embodiments that key elements of the integrated oral appliance 13 interact with upper airway tissue to treat upper airway abnormalities that cause obstructions during sleep. When positioned in a user's mouth (oral cavity), the integrated oral appliance 13 restrains the tongue, preventing the tongue from moving into airway space; moves the mandible in an anterior (forward) direction to open the airway; and reduces tissue pressure to improve airflow. The present invention also provides an air conduit to bypass nasal restrictions and or airway obstructions if they occur.

In an actual reduction to practice an oral appliance of the present invention, the present invention reduced the Apnea Hypopnea Index from 29 (untreated) to 1 and eliminated snoring.

### Additional Embodiments

It should be obvious to those skilled in the art that there are a wide variety of changes that can be made to either the integrated oral appliance 13 or it's method of manufacture. In one alternate embodiment, the present invention can be made with only one of the key features such as with tongue restraint only or with mandible advance only. In another embodiment tongue restraint can be accomplished with attachment to upper dentition or lower dentition using either the upper-tray 1 alone or the lower-tray 2 alone. Tongue restraint can also be accomplished without an internal air conduit 8 if an alternate breathing passage is not required. In other embodiments there can be variations in adjustable features such as adjustable spring force or the type of spring used. In another embodiment element 5 can be replaceable to accommodate wear or made out of a different material that also has non-slip properties. In other embodiments there can be variations in the method of mandible adjustment.

In further embodiments, other attachment, support and/or engagement alternatives for coupling a tongue restraint may be provided to trays coupling to a dentition. Coupling elements to support the tongue restraint may include one or more supports or attachments for placement in one or both of inside and outside the oral cavity. Coupling elements for positioning the tongue restraint in some embodiments may include wires (including but not limited to engaging a portion of dentition), straps, bracings, grips, and the like. It will be appreciated that in such alternative embodiments the supports external or internal of the oral cavity are generally provided for comfortable fitting to the user.

FIG. 5 is a rear right section of an oral appliance illustrating an alternative embodiment for coupling upper tray 1 to lower tray 2 and advancing lower tray 2 relative to upper tray 1. Components 51 and 52 are medical grade hook and loop fasteners fabricated from appropriate polymeric or polymeric materials.

FIG. 6 is a rear right section of an oral appliance illustrating an alternative embodiment for coupling upper tray 1 to lower tray 2 and advancing lower tray 2 relative to upper tray 1. Component 61 is a rigid curved rod attached to component 1 and enables component 62 (attached to 2) to facilitate advancement of component 2. Components 61 and 62 can be made using stainless steel or composite or polymeric materials, or any appropriate material that will functionally perform and will not rust or degrade when exposed to the environment inside the oral cavity.

FIG. 7 is a rear perspective view of an alternative embodiment to engage oral tissue using trays 71 and 72 comprised of Vistamaxx or other appropriate materials, connected with component 73. Component 73 is a support sized to fit the users dental arch and can be made using stainless steel or composite or polymeric materials, or any appropriate material that will functionally perform and will not rust or degrade when exposed to the environment inside the oral cavity.

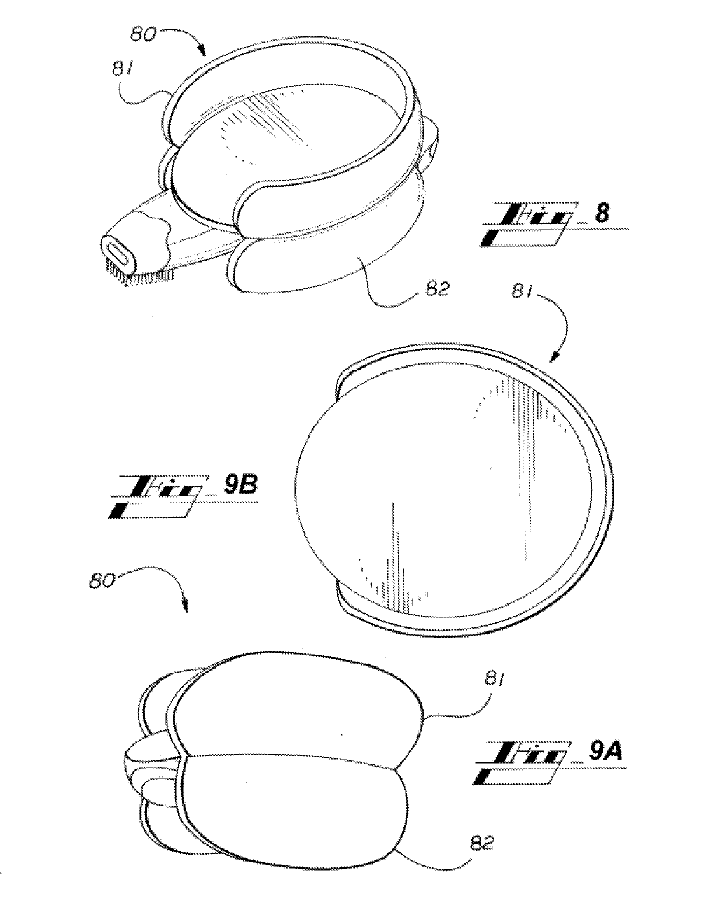

FIG. 8 is a rear perspective view of oral appliance 80 illustrating an alternative embodiment to engage oral tissue including dentition and gingiva and the oral vestibule. Oral appliance 80 comprises elements 81 (upper tray), element 82 (lower tray) (see FIG. 9A), and elements 3 through 8 and 12 (see FIG. 3). Oral appliance 80 does not provide a means to reposition the mandible. Oral appliance 80 does provide an adjustable spring force, and adjustable location for spring element 3. Upper tray 81 and lower tray 82 can be made using flexible rubber materials, Vistamaxx™, composite or flexible polymeric materials, or any appropriate material flexible or semi-flexible material that will functionally perform and will not rust or degrade when exposed to the environment inside the oral cavity. Upper tray 81 also includes a relieved area in the middle of its upper surface to allow space for the frenulum. In an alternative embodiment upper tray 81 and lower tray 82 can also be made so that upper tray 81 and lower tray 82 are adjustable using expandable approaches such as corrugated materials, or heat sensitive materials that can be expanded to fit within the users oral vestibule. Additional embodiments of trays 81 and 82 can incorporate additional means to engage dentition similar to trays 1 and 2 of FIG. 4 to prevent superior/inferior movement of oral appliance 80 if the user's mouth opens during sleep. Trays 81 and 82 can also be made as a single element instead of two elements fastened together.

FIG. 9A is a rear perspective view of oral appliance 80, including trays 81 and 82 and FIG. 9B is a top view of upper tray 81.

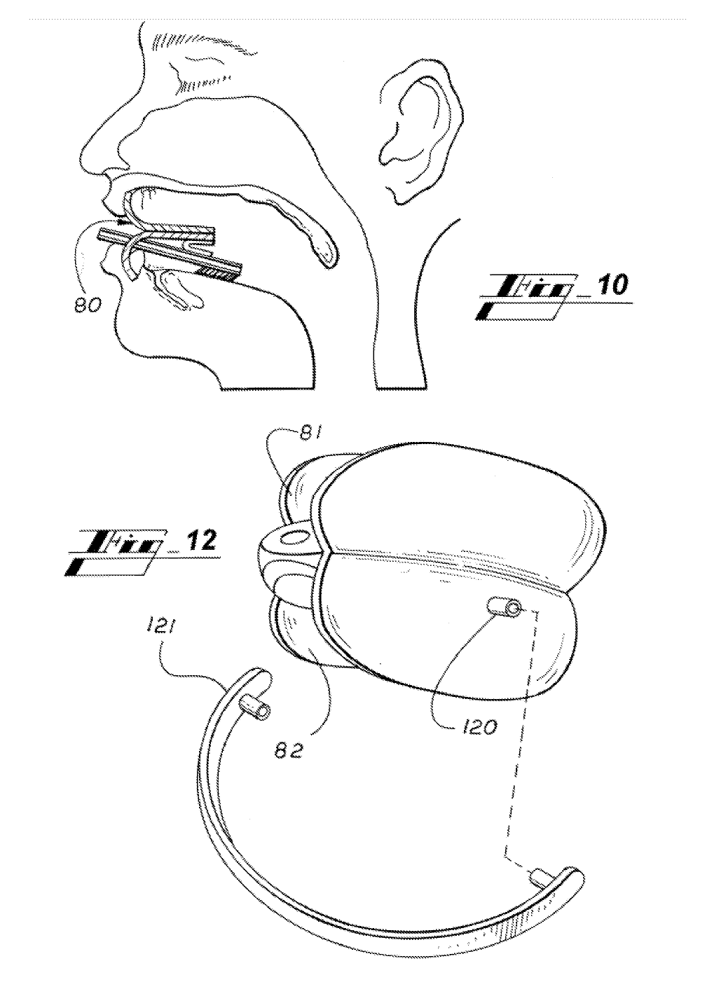

FIG. 10 is a left sectional view of an anatomical cross-section of a human upper airway (similar to FIG. 1), illustrating a cross-section of oral appliance 80 positioned in the oral cavity, engaging the upper and lower dentition, and interacting with the tongue to prevent it from obstructing the airway.

FIG. 11A is a rear perspective view of upper tray 81 and lower tray 82 of an integrated oral appliance 80 comprising an alternative embodiment of a tongue restraint 110, as shown separately in FIG. 11B connected to air conduit 8. FIG. 11C is a perspective view of component 111 illustrates an alternative embodiment to component 110 that engages the tongue.

FIG. 12 is a rear perspective view of upper tray 81 and lower tray 82 of an integrated oral appliance 80 illustrating attachment of connector 120 to tray 82 and additional support strap 121. Strap 121 provides a means to stabilize the oral appliance during sleep and can be made using a variety of flexible materials including silicone rubber, polymeric materials, composite materials and the like. Strap 121 can also be used to comprise other materials, substances, or components such as including, but not limited to, wires, electronics, medications, and the like.

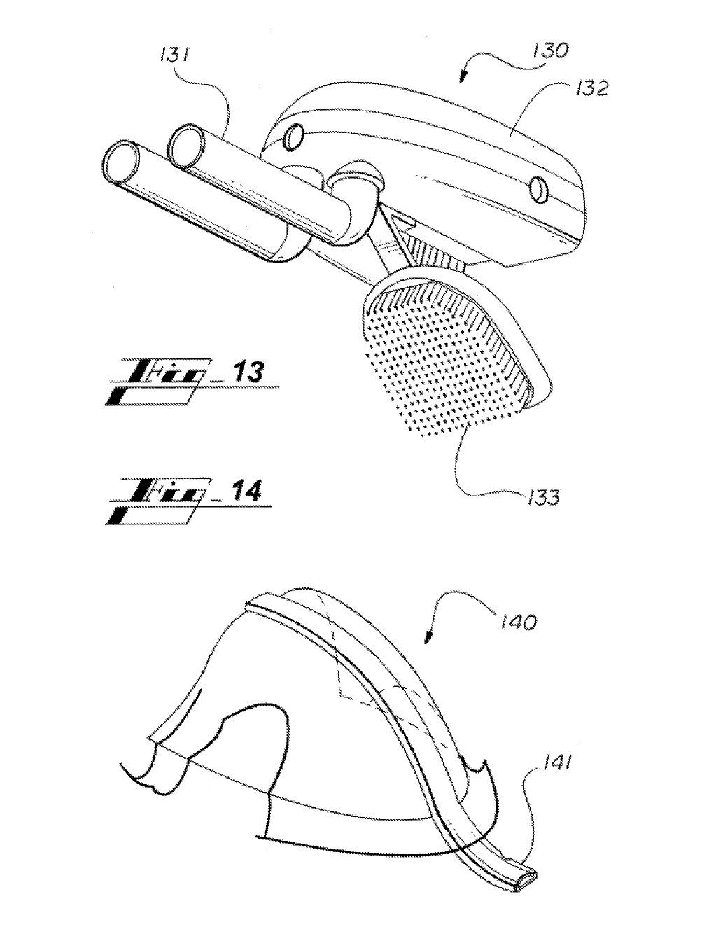

FIG. 13 is a front perspective view of an alternative embodiment of an assembly 130 comprising an air conduit 131, humidification means 132, and adjustable tongue-restraint 133.

FIG. 14 is a front perspective view of an alternative embodiment of a retainer assembly with an air conduit 141. The retainer assembly 140 engages dentition in embodiments of the present invention.

FIG. 15 is an alternative embodiment of a tray 150 to engage dentition comprising an air conduit portion 151A that also a tongue blocking portion 151B that serves to block tongue movement using rigid materials or spring-force.

FIG. 16 illustrates a tray 160 comprising an alternative means to engage dentition or oral tissue using suction cups 161.

FIG. 17 is a perspective view of an alternative means to apply a spring-force to engage the tongue using a coiled spring 172 that can also serve as an air conduit and source of humidification when covered with a fluid absorbent material 171, such as foam.

FIG. 18 is an alternative embodiment of oral appliance 80 comprising electronic components to monitor, screen and report physiological variables related to sleep apnea and device usage. The electronic components, in one embodiment, include a LED 181 and accompanying sensor 182, power supply and electronics compartment 183, sensors 184, and a switch 185. FIG. 18 also illustrates component 5 containing a medication source 186 and component 4 to engage the tongue.

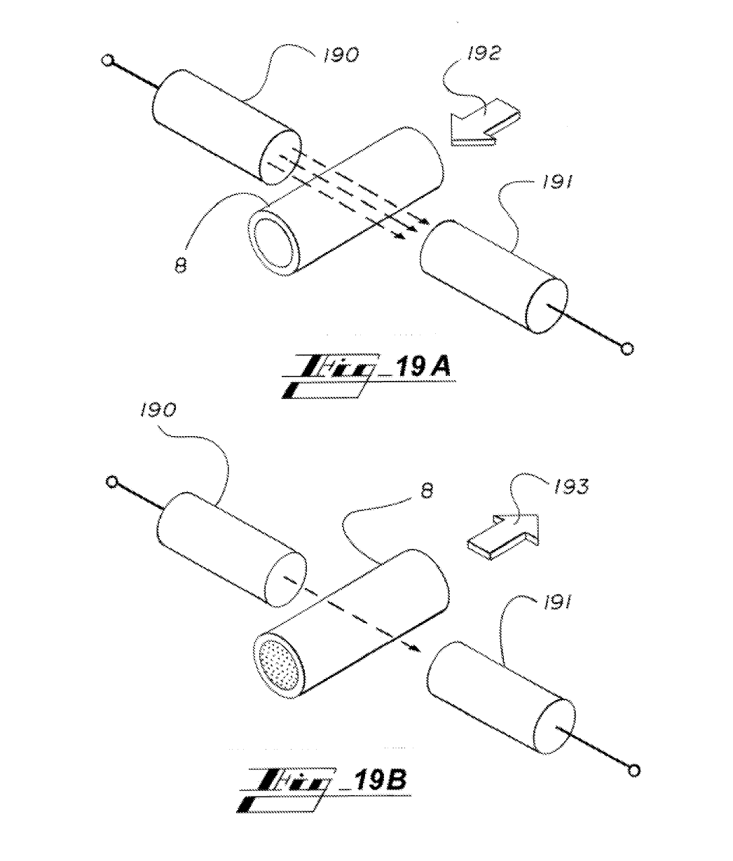

FIGS. 19A and B show electronic means sensing change in light transmission. Optical source 190 transmits light across air conduit 8 that is received by an optical sensor 191 that detects the difference in light transmission between light transmitted across clear inhaled air 192 and the diminished light transmission across exhaled vapor 193 to determine breathing rate within the oral appliance air conduit 8.

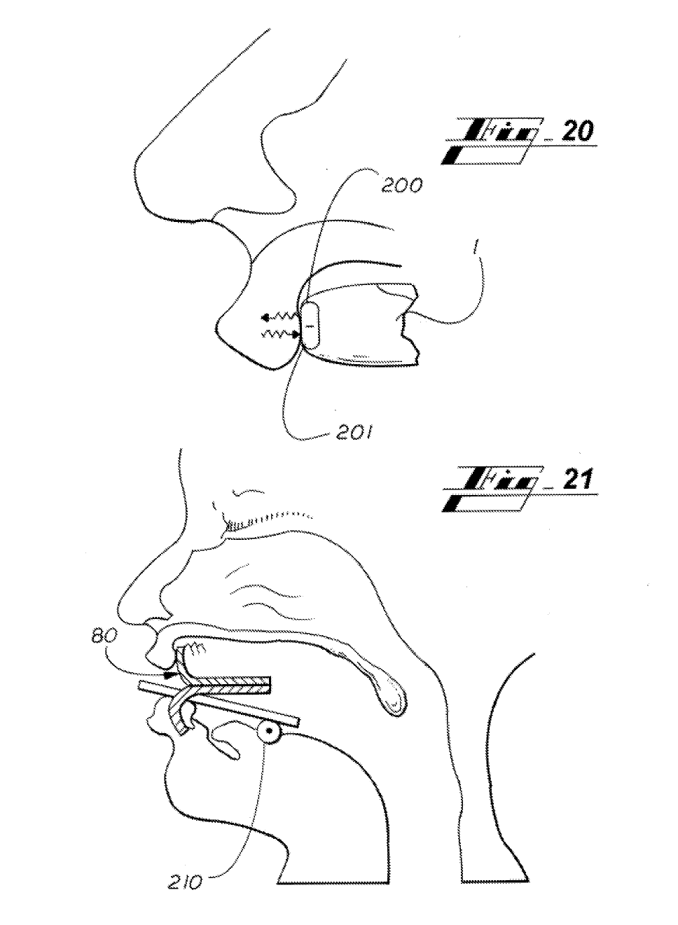

FIG. 20 is an illustration of an optical source 200 and detector 201 mounted within a dental tray 1 that also engages the inside surface of a human or animal lip as part of a system to determine heart rate and or oxygen saturation.

FIG. 21 is a left sectional view of an anatomical cross-section of a human upper airway (similar to FIG. 1), illustrating a cross-section of appliance 80 positioned in the oral cavity, engaging the upper and lower dentition, and interacting with the tongue using electronic means 210 to prevent it from obstructing the airway.

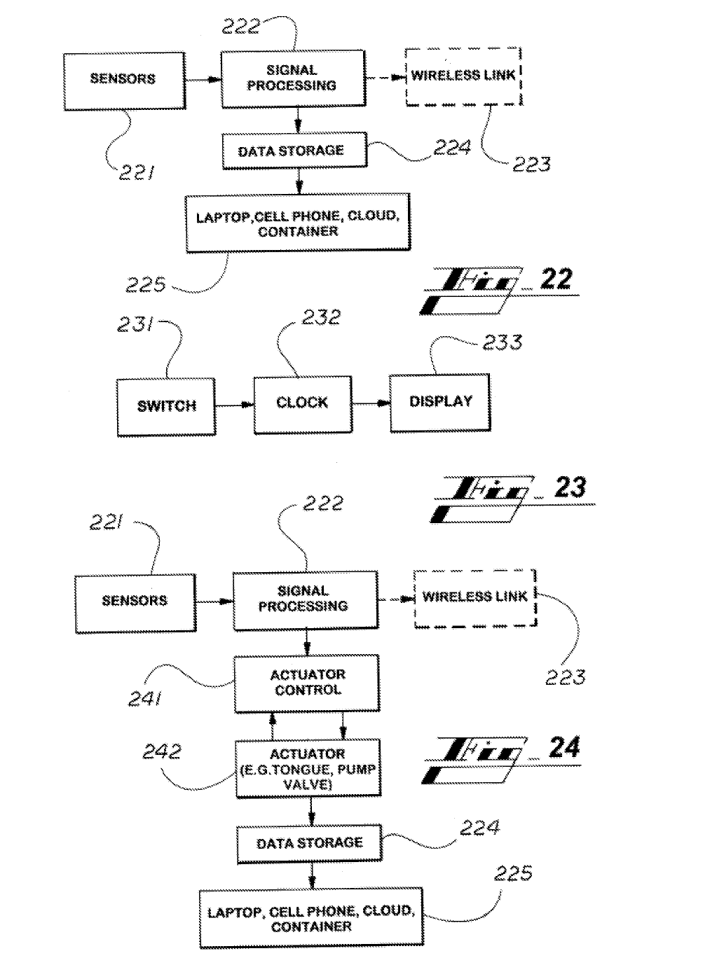

FIG. 22 is a relational block diagram of electronic monitoring of usage and diagnostic screening data from oral appliance sensors such as shown in FIG. 18. In one embodiment sensors 221 are operably coupled to a signal processing device 222, which is operably coupled to a wireless link 223 and data storage 224. The data storage 224 is operably coupled to at least one of a laptop, a cell phone, a cloud, and a container 225.

FIG. 23 is a relational block diagram of alternative embodiment of usage monitoring from interconnection of a clock 232 and display 233 to a switch 231 such as shown in FIG. 18.

FIG. 24 is a relational block diagram of an embodiment of an electronic actuator control 241 including tongue control, motor, pump, valve, and the like, wherein sensors are operably coupled to a signal processing device 222, which is operably coupled to a wireless link 223 and an actuator control. The actuator control is operably coupled to an actuator 242 (e.g. at least one of a tongue, pump, or valve) that is operably coupled to data storage 224. The data storage 224 is operably coupled to at least one of a laptop, a cell phone, a cloud, and a container 225.

In other embodiments of the invention, a tongue restraint is not limited to a passive restraint. In further embodiments, active restraints to the tongue may include electronic, electro-mechanical, and fluidic restraint portions that contact or engage the tongue. In embodiments of the invention, active restraints may be integral of a tongue restraint or operatively coupled to a mechanical restraint to engage a tongue.

Particular embodiments of the present invention have been presented for purposes of illustration and description, and are not intended to be exhaustive or limited to the invention in the form disclosed. Many modifications and variations will be apparent to those skilled in the art. The preferred embodiment was chosen and described in order to best explain the principles of the invention, the basic and practical application, and to enable others of ordinary skill in the art to understand the invention with various embodiments and various modifications as are suited to the particular use contemplated.

Having thus described the invention, what is desired to be protected by Letters Patent is presented in the subsequently appended claims.

We claim:

1. An oral appliance comprising:
an oral cavity engagement device including at least one tray;
a tongue restraint including a tongue contact portion;
an element providing a spring force operatively coupled to the at least one tray and to the tongue restraint, wherein the element providing a spring force and the tongue contact portion are adjustable anteriorly and posteriorly relative to the at least one tray; and
an air conduit configured with a front vent allowing air at atmospheric pressure to enter the air conduit from beyond an anterior end of the at least one tray, and a rear opening of the air conduit at a posterior end of the of the oral cavity engagement device configured to extend to a posterior surface of a tongue.

2. The oral appliance of claim 1, wherein the element providing a spring force applies a spring force to the tongue restraint.

3. The oral appliance of claim 1, wherein the tongue contact portion is rigid and angled downward in relation to the at least one tray.

4. The oral appliance of claim 1, wherein the tongue contact portion includes a slip-resistant surface selected from the group consisting of a surface including projections, a surface including bristles, a meshed surface, a corrugated surface, an irregular surface, a porous surface, a perforated surface, a grated surface, a non-slip surface and an anti-slip surface.

5. The oral appliance of claim 1, wherein the air conduit includes an airflow control valve.

6. The oral appliance of claim 5, wherein the element providing a spring force applies a spring force to the tongue restraint.

7. The oral appliance of claim 5, wherein the tongue contact portion is rigid and angled downward in relation to the at least one tray.

8. The oral appliance of claim 1, wherein the engagement device includes an upper tray and a lower tray.

9. The oral appliance of claim 1, wherein the air conduit includes an absorbent material.

10. The oral appliance of claim 2, wherein a spring force applied to the tongue restraint by the element providing a spring force is force-adjustable.

11. The oral appliance of claim 1, further comprising a sensor disposed in at least one of the oral cavity engagement device and the air conduit.

12. The oral appliance of claim 1, wherein the tongue restraint further includes an actuator.

13. The oral appliance of claim 1, further comprising a light source disposed in at least one of the oral cavity engagement device and the air conduit.

14. The oral appliance of claim 1, further comprising electronics disposed in the oral cavity engagement device.

15. The oral appliance of claim 1, further comprising a strap operatively coupled to the oral cavity engagement device.

16. The oral appliance of claim 1, wherein the element providing a spring force is at least one of a torsion spring, a leaf spring, a coiled spring, a compression spring, an extension spring, a constant force spring, a wire-form spring, pneumatic spring, a elastomeric spring and a cantilevered spring.

17. The oral appliance of claim 1, wherein the at least one tray is configured to engage at least one of a dentition and an oral tissue.

18. The oral appliance of claim 8, wherein the upper tray is adjustably coupled to the lower tray.

19. The oral appliance of claim 18, wherein element providing a spring force is operatively coupled to the upper tray and to the tongue restraint.

20. An oral appliance comprising:
an oral cavity engagement device including at least one tray configured to engage at least one of a dentition and an oral tissue;
a tongue restraint including a tongue contact portion that is slip-resistant;
an element providing a spring force operatively coupled to the at least one tray and to the tongue restraint, wherein the element providing a spring force and the tongue contact portion are adjustable anteriorly and posteriorly relative to the at least one tray; and
an air conduit configured with a front vent allowing air at atmospheric pressure immediately external and adjacent to lips to enter the air conduit from beyond an anterior end of the at least one tray, and a rear opening of the air conduit at a posterior end of the of the oral cavity engagement device configured to extend to a posterior surface of a tongue.
